(19) United States
(12) Patent Application Publication
(10) Pub. No.: US 2012/0283567 A1
(43) Pub. Date: Nov. 8, 2012

(54) ULTRASONIC DIAGNOSTIC APPARATUS AND MEASUREMENT-POINT TRACKING METHOD

(75) Inventor: Tomoaki Chono, Tokyo (JP)

(73) Assignee: HITACHI MEDICAL CORPORATION, Tokyo (JP)

(21) Appl. No.: 13/520,856

(22) PCT Filed: Jan. 20, 2011
(86) PCT No.: PCT/JP2011/050912

§ 371 Date: Jul. 6, 2012

(30) Foreign Application Priority Data
Jan. 29, 2010 (JP) 2010-019124

(51) Int. Cl. A61B 8/08; A61B 8/14

(52) U.S. Cl. 600/447; 600/443

(57) ABSTRACT

An ultrasonic diagnostic apparatus provided with a measurement position setting unit configured to set a region of interest in the cardiac muscle in the ultrasonic image displayed on the display unit, a tracking calculation unit configured to track the movement of the cardiac muscle at a plurality of measurement points in the region of interest, and a physical quantity calculating unit configured to calculate a specific physical quantity on the basis of the results of the tracking. The calculated specific physical quantity is displayed on the display unit. The tracking calculation unit tracks the measurement points using a plurality of first segmenting lines along the epicardium and the endocardium of the cardiac muscle and a plurality of second segmenting lines that are orthogonal to the second segmenting lines.

### FIELD OF THE INVENTION

The present invention relates to an ultrasonic diagnostic apparatus, in particular to a technique for tracking the movement of biological tissue on the basis of an ultrasonic image of biological tissue of an object to be examined, which calculates and displays a specific physical quantity correlated with the movement or property of biological tissue based on the tracking results.

### DESCRIPTION OF RELATED ART

An ultrasonic diagnostic apparatus transmits ultrasonic waves to the inside of an object via an ultrasonic probe and receives the reflected echo signals of ultrasonic waves in conformity to the structure of the biological tissue from the inside of the object, so as to construct an ultrasonic image (for example, an ultrasonic tomographic image such as a B-mode image) and displays the image for the use of diagnosis.

In recent years, a technique has been used for diagnosis which tracks the movement of biological tissue on the basis of an ultrasonic image, and calculates a specific physical quantity correlated with the movement or property of the biological tissue (hereinafter referred to merely as physical quantity) on the basis of the tracking result. For example, when a target for diagnosis is cardiac muscle, a technique is known which calculates and displays a specific physical quantity such as the moving velocity of cardiac muscle or the strain (distortion) which is the property of cardiac muscle tissue on the basis of the tracking result, for diagnosis of a heart disease such as ischemic heart disease.

As for the method of tracking biological tissue in ultrasonic diagnostics, techniques such as tissue Doppler and speckle tracking have been proposed. Speckle tracking in particular, is capable of tracking the positions where biological tissue has been moved and quantifying the distortion of a region in a living body which is related to the biological tissue without depending on the direction of ultrasonic beams, and is applied, for example to track the movement of the cardiac muscle of an object.

As for the method of tracking the movement of cardiac muscle, as disclosed in Patent Document 1, a technique is known which executes a tracking process by extracting a plurality of traceable points from an ultrasonic image, and calculates a specific physical quantity on the basis of the movement information on the tracked points. Also, as disclosed in Non-patent Document 1, a technique is known which measures cardiac muscle of the left ventricle by segmenting it into 17 regions and makes a diagnosis of a disease based on the respective measurement values.

### PRIOR ART DOCUMENTS

### Patent Document

Patent Document 1: Japanese Patent No. 4060615

### Non-Patent Document

Non-patent Document 1: Robert M. Lang, et al., Recommendations for Chamber Quantification, Journal of American Society of Echocardiography, Vo. 18, No. 12

However, the conventional techniques disclosed in the above-mentioned Patent Document 1 and Non-patent Document 1 do not consider the measurement of expansion and contraction of cardiac muscle. Effective reflection of expansion and contraction of cardiac muscle facilitates the measurement values of cardiac muscle to be a target for comparison to make appropriate diagnosis.

In the method of tracking the movement of cardiac muscle, it is common to set a region of interest in the cardiac muscle on an ultrasonic image and track the cardiac muscle in a plurality of measurement points (tracking points) in the region of interest. In this regard, the conventional techniques do not consider the setting a plurality of measurement points along the expansion/contraction direction of cardiac muscle (for example, the direction along the endocardium and the epicardium of cardiac muscle).

Therefore, for example, in the case that the variation of distance between the adjacent measurement points caused by expansion/contraction of cardiac muscle, the expansion and contraction of the cardiac muscle may not be accurately reflected to the respective measurement points. Also, since the distance from the respective measurement points to the endocardium or the epicardium of the cardiac muscle is not constant, even when the variation of the distance caused by expansion and contraction of the cardiac muscle is measured, the respective measurement values may not be suitable reference for comparison to make a proper diagnosis.

Given this factor, the objective of the present invention is to provide the ultrasonic diagnostic apparatus and the measurement-point tracking method capable of measuring expansion and contraction of the cardiac muscle, which facilitates accurate reflection of expansion and contraction of cardiac muscle to the respective measurement points for making a proper diagnosis.

### BRIEF SUMMARY OF THE INVENTION

In order to achieve the above-described objective, in the present invention, a measurement position setting unit sets a region of interest in the cardiac muscle on an ultrasonic image which is displayed on a display unit, and a tracking calculation unit tracks a plurality of measurement points by a plurality of first segmenting lines along the epicardium and the endocardium of the cardiac muscle in a plurality of measurement points of the region of interest and a plurality of second segmenting lines that are orthogonal to the plurality of first segmenting lines.

In concrete terms, the ultrasonic diagnostic apparatus of the present invention comprises:

an ultrasonic probe configured to transmit/receive ultrasonic waves to/from an object;

an ultrasonic signal generating unit configured to generate ultrasonic signals on the basis of the reflected echo signals, that are received by the ultrasonic probe, of the cross section of the tissue including the cardiac muscle of the heart of the object;

an ultrasonic image generating unit configured to generate an ultrasonic image on the basis of the ultrasonic signals;

a display unit configured to display the ultrasonic image;

a measurement position setting unit configured to set a region of interest in the cardiac muscle on an ultrasonic image which is displayed on the display unit;

a tracking calculation unit configured to track the movement of cardiac muscle in a plurality of measurement points of the region of interest; and

a physical quantity calculating unit configured to calculate a specific physical quantity based on the tracking result,

wherein:

the calculated specific physical quantity is displayed on the display unit; and

the tracing calculation unit tracks the plurality of measurement points by a plurality of segmenting lines along the epicardium and the endocardium of the cardiac muscle and a plurality of second segmenting lines that are orthogonal to the plurality of first segmenting lines.

Also, the measurement-point tracking method of an ultrasonic diagnostic apparatus related to the present invention:

transmits/receives ultrasonic waves to/from an object via an ultrasonic probe;

generates ultrasonic signals by an ultrasonic signal generating unit on the basis of the reflected echo signals, received by the ultrasonic probe, of the cross section of the tissue including the cardiac muscle of the heart of the object;

generates an ultrasonic image by an ultrasonic image generating unit on the basis of the ultrasonic signals;

displays the ultrasonic image by a display unit;

sets a region of interest by a measurement position setting unit in the cardiac muscle on an ultrasonic image which is displayed on the display unit;

tracks the movement of cardiac muscle by a tracking calculation unit in a plurality of measurement points in the region of interest;

calculates a specific physical quantity a physical quantity calculating unit on the basis of the tracking result; and

displays the calculated specific physical quantity on the display unit,

including a step of tracking the plurality of measurement points by the tracking calculation unit, using a plurality of first segmenting lines along the epicardium and the endocardium of the cardiac muscle and a plurality of second segmenting lines that are orthogonal to the plurality of first segmenting lines.

### EFFECT OF THE INVENTION

In accordance with the present invention, it is possible to measure expansion and contraction of cardiac muscle which facilitates accurate reflection of expansion and contraction of cardiac muscle to the respective measurement values for making a proper diagnosis.

### BRIEF DESCRIPTION OF THE DRAWINGS

FIG. 1 is a block diagram showing the general configuration of the ultrasonic diagnostic apparatus in the present embodiment.

FIG. 2 is a flowchart showing the processing flow of the ultrasonic diagnostic apparatus in the present embodiment.

FIG. 3 is an example of a display screen in the ultrasonic diagnostic apparatus.

FIG. 4 is an example of a display screen showing measurement results.

FIG. 5 is another example of a display screen in the ultrasonic diagnostic apparatus.

FIG. 6 is an enlarged view of a region of interest.

FIG. 7 is an example of the measurement method in a 3-dimensional ultrasonic image.

### DETAILED DESCRIPTION OF THE INVENTION

An embodiment of the ultrasonic diagnostic apparatus to which the present invention is applied will be described below. In the following description, the same function parts are represented by the same reference numerals, and the duplicative description thereof is omitted.

(Configuration of Ultrasonic Diagnostic Apparatus)

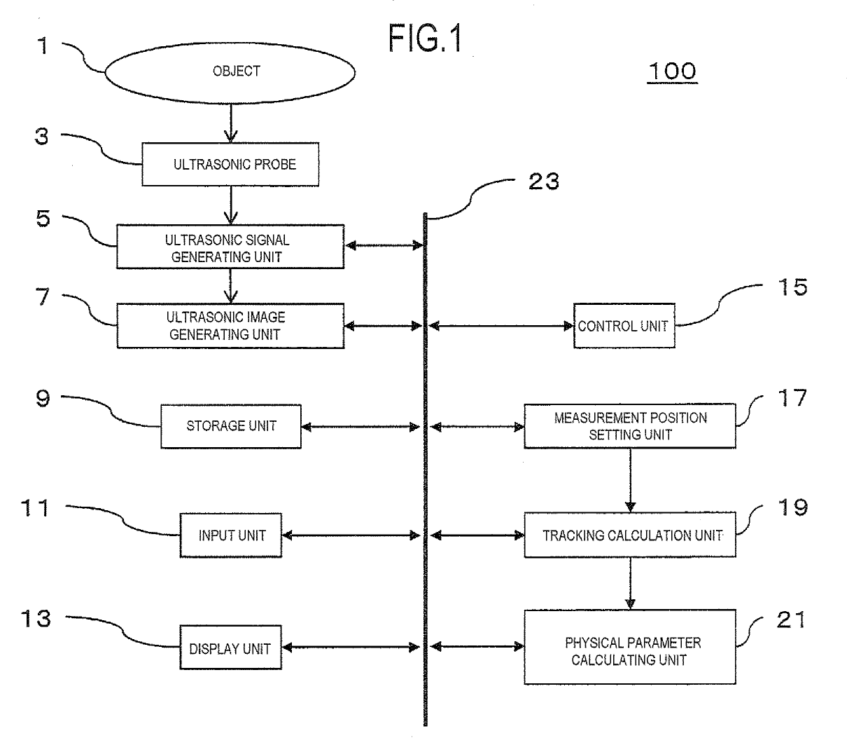

FIG. 1 is a block diagram showing the general configuration of the ultrasonic diagnostic apparatus in the present embodiment. As shown in FIG. 1, an ultrasonic diagnostic apparatus 100 of the present embodiment comprises an ultrasonic probe 3 configured to transmit/receive ultrasonic waves to/from an object 1, an ultrasonic signal generating unit 5 configured to generate ultrasonic signals on the basis of the reflected echo signals measured by the ultrasonic probe 3, an ultrasonic image generating unit 7 configured to generate an ultrasonic image on the basis of ultrasonic signals, a storage unit 9 configured to store the generated ultrasonic image or various programs for controlling the ultrasonic diagnostic apparatus, an input unit 11 to be the input interface, a display unit 13 to be the output interface, a control unit 15 configured to control the respective units of the ultrasonic diagnostic apparatus, a measurement position setting unit 17 configured to set a region of interest in biological tissue of a diagnostic target region on the ultrasonic image which is displayed on the display unit 13, a tracking calculation unit 19 configured to track the movement of biological tissue in a diagnostic target region in a plurality of measurement points of a region of interest, a physical quantity calculating unit 21 configured to calculate a specific physical quantity on the basis of the tracking result, and a system bus 23 configured to connect the respective units.

The ultrasonic probe 3 transmits/receive ultrasonic waves to/from the object 1, provided with a scanning method such as a linear type, convex type or sector type. These types of ultrasonic probe can be provided with configuration and function in which transducers are arrayed one-dimensionally to acquire 2-dimensional signals, transducers are arrayed 2-dimesionally to acquire 3-dimensional signals, or transducers are arrayed one-dimensionally to acquire 3-dimensional signals by mechanically executing special scanning.

The ultrasonic signal generating unit 5 transmits and receives ultrasonic waves that are converted into electric signals between the unit and the ultrasonic probe 3. It receives the information on the power or timing of transmission and reception from the control unit 15, so that the transmission and reception are controlled to acquire desired ultrasonic waves. The ultrasonic signal generating unit 5 also executes signal processing via a phasing and adding unit or an amplifier circuit on the signals received from the ultrasonic signal transmission/reception unit in conformity to the imaging setting, and acquires the shaped ultrasonic signals. These signals are stored in the storage unit 9 to be used for the subsequent measurement.

The ultrasonic image generating unit 7 generates an image of biological tissue of the object 1. The ultrasonic signals passed through the ultrasonic signal generating unit are input, and an ultrasonic image based on the imaging setting of the apparatus is generated. These signals are stored in the storage unit 9 for using the subsequent measurement.

The storage unit 9 stores the programs for operating various systems which configure the ultrasonic diagnostic apparatus 100 and data such as signal data, image data and measurement data, and reading and writing are executed in response to the processing.

The input unit 11 is the interface for executing various operations of the diagnostic apparatus. It is an input device such as a keyboard, trackball, switch or dial, and is used for performing operation to obtain images, specifying a region of interest of biological tissue, or executing various measurement settings.

The display unit 13 displays a region of interest, measurement values, and an ultrasonic image on a screen, or outputs the measurement values as a measurement report. The control unit 15 controls the entire system. For example, device such as a CPU is used as the control unit.

The measurement position setting unit 17 sets a region of interest on an ultrasonic image. For example, it segments the region of interest into a meshed pattern, and executes the process to set the intersecting points of segmenting lines as the measurement points. The measurement position setting unit 17 will be described later in detail. On the ultrasonic screen, the ultrasonic image which is read out from the storage unit 9 is displayed. Since the images are stored in time-series order, the frame of a desired time phase can be selected and displayed using an input device. If an image is obtained by synchronizing with biological signals (for example, an electrocardiogram), it may also be configured, for example so that an image of the R-wave phase of an electrocardiogram can be automatically selected.

The tracking calculation unit 19 performs calculation for tracking the movement of biological tissue using the ultrasonic signal near the position of the measurement point or the amplitude information of the image, and calculates the displacement thereof. The information on the ultrasonic signal and the image which is stored in the storage unit 9 is read in and used for the calculation. The starting frame and the ending frame of the tracking calculation may be set by a user using an input device, or if an image is obtained by synchronizing with a biological signal (for example, an electrocardiogram), the frame group may be automatically limited, for example in the range from an R-wave to the next R-wave of an electrocardiogram.

The physical calculation unit 21 calculates the physical quantity such as velocity, strain, area and volume as time-series information on the basis of the displacement, in accordance with the measurement items appointed by an examiner using the input device. The calculated physical quantity is displayed by the display unit 13 on an ultrasonic image by pseudocolor display or numeric values, or output as a measurement report file. The processing flow and characteristic configuration of the ultrasonic diagnostic apparatus in the present embodiment will be described in detail for each embodiment.

### Embodiment 1

The first embodiment is an example, by citing a cardiac short axis view, of spatial, temporal and consecutive measurement of the inside of cardiac muscle and calculation of a measurement value thereof for each local portion. Here, the measurement target will be described as a 2-dimensional ultrasonic signal and a 2-dimensional ultrasonic image.

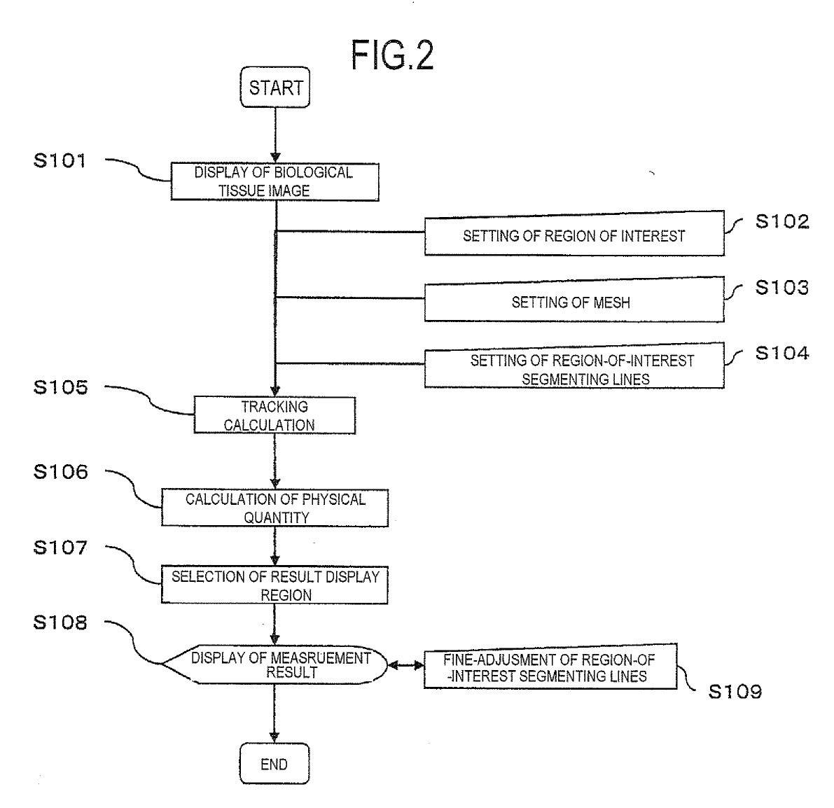

FIG. 2 is a flowchart showing the processing flow of the ultrasonic diagnostic apparatus in the present embodiment. As shown in FIG. 2, the examiner first images and displays an ultrasonic image of biological tissue (cardiac muscle) (S101). The ultrasonic transmitting/receiving surface of the ultrasonic probe 3 is applied to an object 1, and a region including the measurement target is imaged. In order to execute tracking, images are obtained for a certain period of time. If biological signals (for example, electrocardiogram) are input, the image may also be obtained by synchronizing with the input signals. For example, one heartbeat from an R-wave to the next R-wave of the electrocardiogram may be automatically obtained, or the starting frame and the ending frame may be specified with respect to the frame group in a certain interval after the image is obtained. While the specified frame group is stored in the storage unit 9, when the tracking calculation is to be executed using ultrasonic signals, the ultrasonic signals are also stored in the storage unit 9 in the same manner as the ultrasonic images to be read out for the calculation.

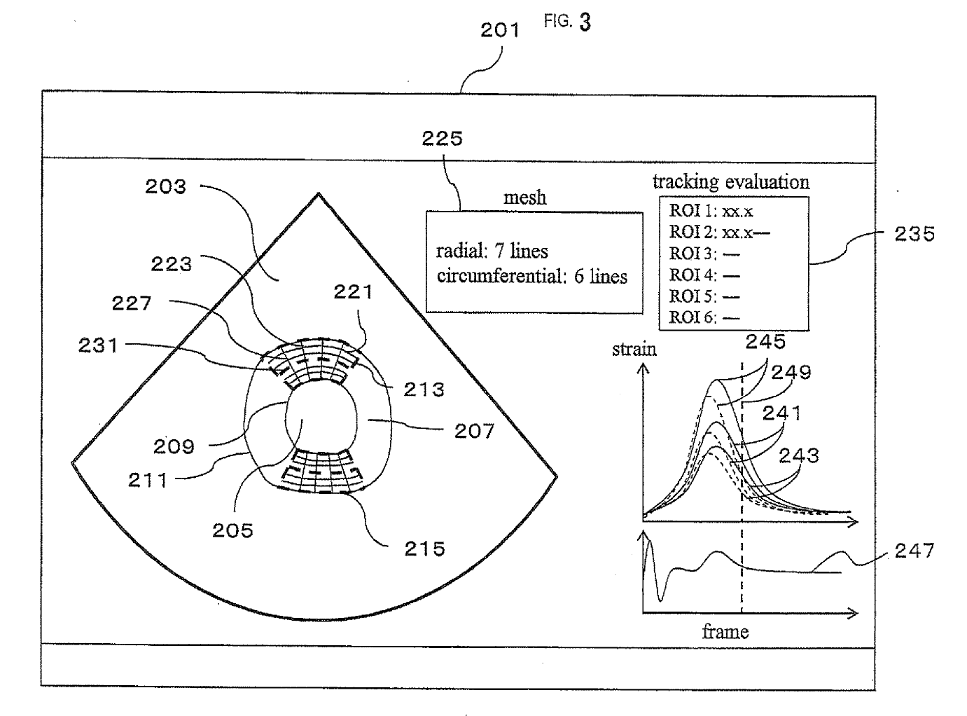

FIG. 3 is an example of a display screen of the ultrasonic diagnostic apparatus. On a display screen 201 of the ultrasonic diagnostic apparatus, an ultrasonic image 203 is displayed on the left side of the screen, and the cardiac short axis view is displayed here. More specifically, on the ultrasonic image 203, a short axis view is displayed including a heart chamber 205 of a heart of an object and a cardiac muscle 207 of a doughnut shape surrounding the heart chamber 205. Further, an endocardium 209 exists on the border between the cardiac muscle 207 and the heart chamber 205 along the circumferential direction, and an epicardium 211 exists on the border between the cardiac muscle 207 and the other tissue on the outer circumferential side of the cardiac muscle 207 along the circumferential direction.

Next, the examiner sets a region of interest by the measurement position setting unit 17 (S102). The setting is executed using an input device on the ultrasonic image displayed in S101. FIG. 3 shows an example of a screen for setting a region of interest. A short axis view is depicted on the screen, and zonal regions of interest 213 and 215 are set on the image thereof. As for the setting method, an existing automatic setting method based on the region segmentation method may be used, or the contour of cardiac muscle may be manually traced. While zonations to surround a cardiac muscle are used here, the setting method of the present invention is not limited thereto. The examiner may also set a region of interest in a desired target portion itself, for example a tumor or a part of the inside of cardiac muscle, without surrounding the region by the border of cardiac muscle. Also, the number of regions of interest may be set as one or plural numbers. For example, in the doughnut-shaped cardiac muscle in the cardiac short axis view, a plurality of partial regions in cardiac muscle can be separated and set as regions of interest. In this manner, a specific physical quantity of cardiac muscle can be compared and observed in plural regions of interest. In the example of FIG. 3, a first region of interest 213 which is close to the transmission/reception surface of the ultrasonic probe 3 and a second region of interest 215 which is on the far side are set as separate regions of interest, with the heart chamber 205 interposed therebetween.

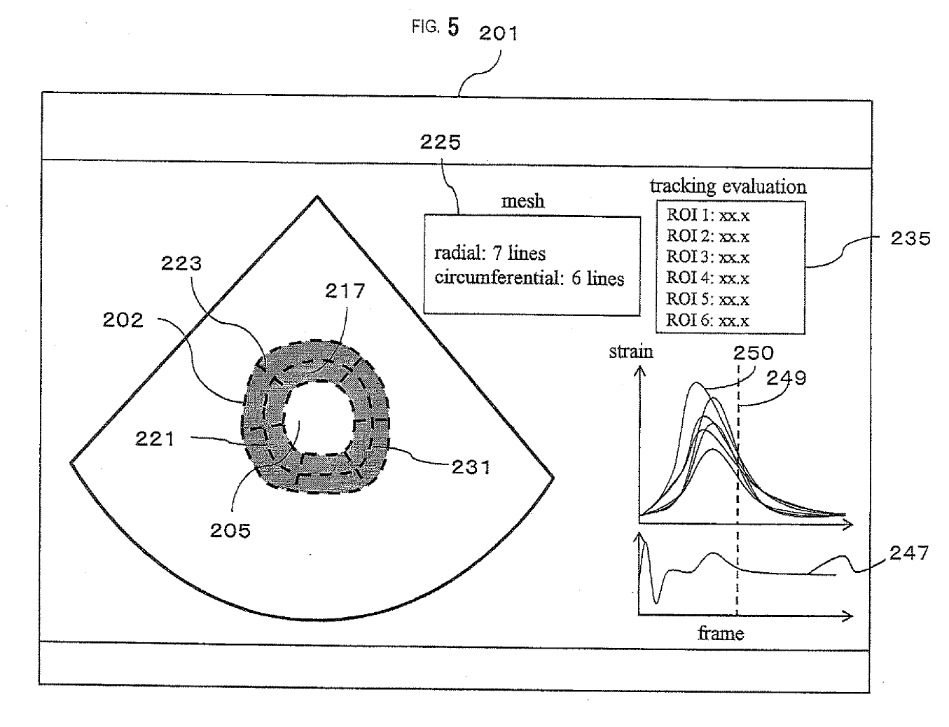

FIG. 5 is another example of a display screen of the ultrasonic diagnostic apparatus. As shown in FIG. 5, by setting a doughnut-shaped region of interest 217 which surrounds the doughnut-shaped entire cardiac muscle, and the set region of interest may be divided into six segments based on the 17-segment method which is recommended by ASE (American Society of Echocardiography).

Next, the examiner sets the method of mesh division by the measurement position setting unit 17 (S103). The mesh division method can be adapted to different measurement items of target tissue. Since cardiac muscle is the target in this case, as shown in FIG. 3, the directions for segmenting the regions into meshed pattern are set in the circumferential direction and the radial direction. That is, the objective of segmentation is to measure the expansion and contraction in the circumferential direction and the radial direction of the cardiac muscle. In other words, the regions of interest are segmented into meshed pattern by a plurality of first segmenting lines 221 along the epicardium 211 and the endocardium 209 of the cardiac muscle and a plurality of second segmenting lines 223 that are orthogonal to the plurality of first segmenting lines 221, and a plurality of intersecting points of the first segmenting lines 221 and the second segmenting lines 223 are set as measurement points respectively. The density of the mesh, i.e. the number of the first segmenting lines 221 and the second segmenting lines 223 with respect to a certain region can be arbitrarily adjusted.

As the number of mesh segmentation increases, measurement can be performed in more detail and smoother luminance variation can be depicted in pseudocolor display. The measurement may also be executed on the basis of the distance between the inner membrane side and the outer membrane side as the conventional method without performing any segmentation. The setting of the number of segmentation may be freely selected by the examiner using an input device. The set values are displayed on a mesh setting display 225.

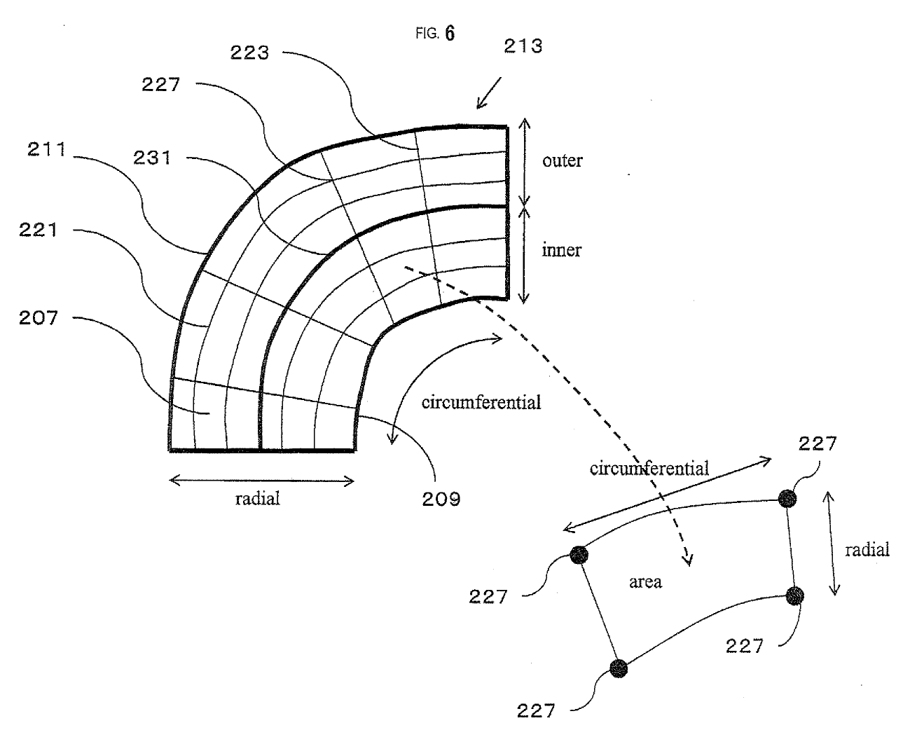

Here, seven first segmenting lines 221 are used for segmenting the cardiac muscle in the radial direction, and six second segmenting lines 223 are used for segmenting the cardiac muscle in the circumferential direction. These segmenting lines cross each other, and the intersecting points are set as measurement points 227. FIG. 6 is an enlarged view of a region of interest. The lower right part of FIG. 6 is a view showing a segment which is abstracted from a region of interest. The measurement points 227 are set on the contact points of the segments, i.e. on the intersecting points of the meshed pattern. Therefore, 7×6=42 of measurement points are set in this case.

Next, the examiner sets a region-of-interest segmenting lines 231 by the measurement position setting unit 17 (S104). The region-of-interest segmenting lines 231 are to be set when the inside of a region of interest is desired to be further segmented into groups (a region of interest is divided into two groups in this example). While the region-of-interest segmenting line 231 is set at approximately intermediate position between the endocardium and the epicardium since it is expected that the property of cardiac muscle is different in the endocardium and the epicardium as shown in FIGS. 3˜6, it should be configured as freely settable in conformity to the target of measurement. For example, the region-of-interest segmenting line 231 can be set to surround a tumor.

Also, while the case that one of the plurality of first segmenting lines 221 that are set in a region of interest is set as the region-of-interest segmenting line 231 is exemplified in the present embodiment, the present invention is not limited thereto. For example, one of the plurality of second segmenting lines 223 can be set as a region-of-interest segmenting line. Also, a line other than the first segmenting lines 221 or the second segmenting lines 223 can also be set as a region-of-interest segmenting line.

Next, the ultrasonic diagnostic apparatus measures the movement of a measurement-point group by the tracking calculation unit 19 (S105). The tracking calculation unit tracks a plurality of measurement points using the first segmenting lines 221 and the second segmenting lines 223. That the tracking calculation unit tracks the plurality of measurement points that are set on the basis of the first segmenting lines 221 and the second segmenting lines 223. For example, the intersecting points of the first segmenting lines 221 and the second segmenting lines 223 may be respectively set as measurement points as described above, or a predetermined position within the respective lattices formed by the first segmenting lines 221 and the second segmenting lines 223 may also be set as measurement points. As for the tracking method, a commonly-known method can be used such as the correlation method or optical flow method. The target signal is the ultrasonic signal or ultrasonic image stored in the storage unit 9.

Since the measurement points are set in high density, the method for improving the resolution may also be used.

For example, the tracking calculation can be adopted by presumptively improving the resolution of a target image. Also, the tracking calculation can be adopted while recursively changing the block size for a block matching. The method of adopting the tracking calculation in which the correlation method and the optical flow method are combined can also be used. The time phase for the measurement is the frame group of an ultrasonic image acquired and set in S101. As the tracking result, the data of positional coordinates in the respective measurement points of each frame is obtained.

Next, the ultrasonic diagnostic apparatus executes calculation of physical quantity by the physical quantity calculating unit 21 (S106). As shown in the lower right part of FIG. 6, with respect to the group of measurement points 227, the physical quantity is calculated based on the distance between two adjacent points. For example, the distance between the two points and the rate of change (strain) from the initial distance are calculated. The radial strain is calculated in the radial direction, and the circumferential strain is calculated in the circumferential direction. Also, the area strain may be calculated on the basis of the change of area which is framed in by the four points. In this manner, the physical quantity of the respective small regions in the meshed pattern is calculated. Further, the average value of these physical quantities in the entire meshed pattern is calculated as the measurement value of the region of interest. Also, the average value is calculated in the epicardium and the endocardium that are divided by the region-of-interest segmenting line 231, and set as the measurement values respectively.

Next, the examiner or the apparatus selects a result display region (S107). The selection criterion is the accuracy of tracking. Here, the evaluation value is calculated for self-evaluation of the tracking accuracy. Generally, the tracking accuracy tends to be lowered as the resolution is increased. Especially ultrasonic images have tendency to be under adverse condition with respect to the tracking calculation due to existence of artifacts or low resolution. Therefore, the tracking cannot always be calculated accurately depending on the condition. As for the evaluation value, the method based on the error-vector detection is used.

For example, since there are many measurement points, the quantity of error-vectors is digitalized using the method of determining that the displacement of a vector at a certain measurement point is different (in error) compared to the surrounding displacement vectors.

Since the evaluation value in the respective measurement points can be obtained, the values may also be calculated either in the entire region of interest or by dividing the region of interest using the region-of-interest segmenting line into the endocardium side and the epicardium side. The examiner selects a result display area while checking the magnitude of evaluation value so that the region of interest with low measurement accuracy will not be displayed. Also, the apparatus may automatically make a selection on the basis of the previously set threshold value.

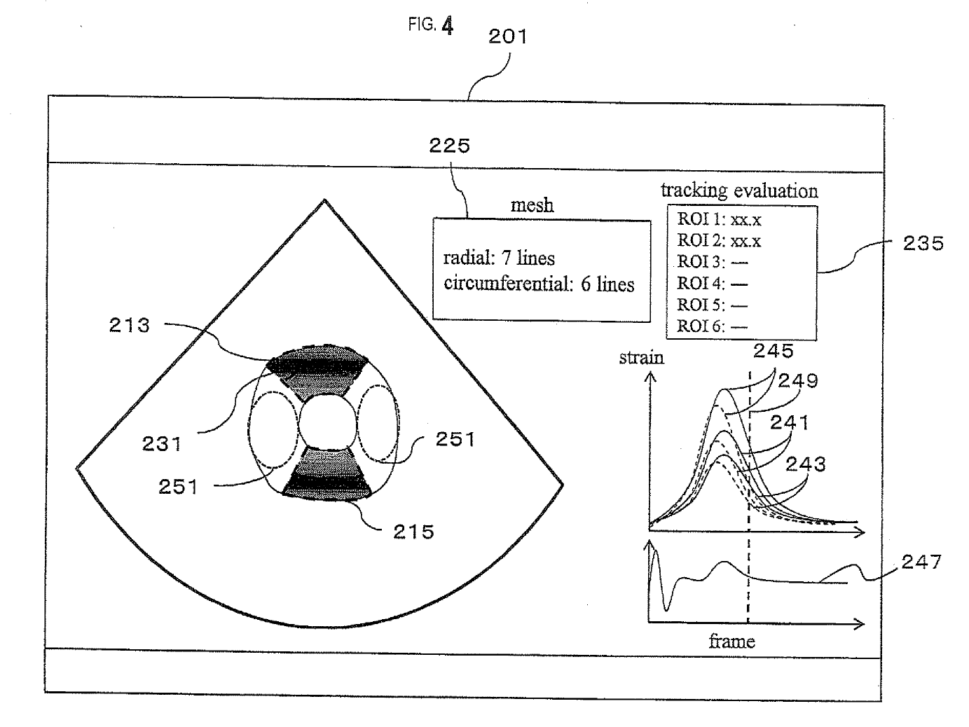

Next, the apparatus displays the measurement result by the display unit 13 (S108). FIG. 4 is an example of a display screen showing the measurement result. The physical quantity is converted into the luminance value for pseudocolor display. These luminance values are superimposed over the meshed pattern of the region of interest and interpolated for smooth luminance variation, so as to color the entire region of interest. In FIG. 4, the coloring is performed in all of the regions of interest 213 and 215. The self-evaluation value of the tracking is displayed on the chart of a tracking self-evaluation result 235.

The examiner checks the magnitude of these numeric values and determines whether to adopt the measurement result. A graph is displayed on the right side of FIG. 4. The graph is displayed for each local region in the region of interest, for example as a strain value 241 of the entire region of interest, a strain value 243 of the endocardiam-side region and a strain value 245 of the epicardium-side region. A biosignal 247 is also displayed. A time phase bar 249 is further provided to the time phase of the currently displayed image for movie display. In the case that six regions of interest are set as shown in FIG. 5, each region of interest is pseudocolor-displayed and the number of graphs 250 to be displayed will be the same as the number of regions of interest. Here, the graphs of the first region of interest 213 are denoted by solid lines, and the graphs of the second region of interest 215 are denoted by dotted lines. It may also be operated, when the tracking self-evaluation result 235 is observed on a certain region of interest and determined that the measurement result thereof is not to be adopted, not to color-display only that region of interest.

Next, the examiner fine-adjusts the region-of-interest segmenting line 231 by the measurement position setting unit (S109). The examiner adjusts the position of the region-of-interest segmenting line while observing the result of movement tracking. The measurement result in the local regions changes when the region-of-interest segmenting line is adjusted, thus the adjustment is made so that this change is reflected on the graph and the measurement result of a desired position can be obtained while observing the graph.

As described above, in accordance with the present embodiment, it is possible to set a region of interest in conformity with the property of biological tissue, and the movement of the inside of biological tissue can be tracked by setting measurement points in a meshed pattern. By segmenting a region of interest by the region-of-interest segmenting line 231 and setting the segmented regions as local regions or pseudocoloring the measurement values, the difference of the local property in the inside of biological tissue can be easily discriminated. Also, measurement accuracy can be improved by segmenting the region of interest into plural regions such as a first region and a second region so as to set them at measurement positions in conformity to the effected area of an object or set them at the places where sufficient image quality can be provided. Also, by segmenting the region of interest and setting only necessary portions for measurement, calculation amount can be reduced compared to tracking the entire cardiac muscle, thereby improving the examination efficiency.

More specifically, in the cardiac short axis view, when a region of interest is set on the cardiac regions of a doughnut-shaped cardiac muscle other than the region which is close to the transmitting/receiving surface of the ultrasonic probe and the region which is away from the surface, with the heart chamber interposed therebetween (for example, cardiac muscle regions 251 in FIG. 4), the image quality of an ultrasonic image will not be sufficient due to influence of noise, etc., which could lead to the lowering of accuracy in the movement tracking. Therefore, even when the physical quantity is calculated in these cardiac muscle regions, the calculated quantity may lack credibility. If the region of interest is set as a doughnut-shaped pattern despite the above-described tendency, the measurement will be executed even in the regions which lack credibility, thus is not preferable in view of calculation efficiency. In view of this, by segmenting the doughnut-shaped cardiac muscle in a cardiac short axis view and setting a region which is close to the transmitting/receiving surface of an ultrasonic probe and a region which is away from the surface with the heart chamber interposed therebetween as shown in FIG. 3, it is possible to improve the efficiency in calculation process and to enable contrast observation between the region which is close to the transmitting/receiving surface of the ultrasonic probe and the region which is away from the surface, with the heart chamber interposed therebetween.

Also, measurement points are appropriately set in the present embodiment, in conformity to the property or expansion and contraction of the cardiac muscle. That is, multiple measurement points are set along the main expanding and contracting directions (the circumferential direction and the irradiating direction (radial direction)) of the cardiac muscle. In accordance with the present embodiment, since measurement points are set along the expanding and contraction direction of the cardiac muscle as described above, in the case, for example that the variation of distance is measured between the adjacent measurement points on a first segmenting line 221 or between the adjacent measurement points on a second segmenting line 223, the measurement values are useful for making an accurate diagnosis since the property of the respective regions in the cardiac muscle is appropriately reflected to the measurement values. Also, the all of the distances from the endocardium 209 and the epicardium 211 become constant in relation to the respectively set plural measurement points on the plurality of first segmenting lines 221. Therefore, for example, in the case that the variation of distance is measured from the respective measurement points on a certain first segmenting line 221 to the endocardium 209 or the epicardium 211, the property of the respective regions in the cardiac muscle can be appropriately compared by comparing the respective measurement values, thus the measurement results are useful for making an accurate diagnosis. In this manner, in accordance with the present embodiment, it is possible to measure expansion and contraction of cardiac muscle to be reflected to the measurement values of physical quantity, which enables accurate comparison of measurement values for making a proper diagnosis.

### Embodiment 2

The second embodiment is the method, citing an example of a cardiac short axis view, which spatially, temporally and consecutively measures the inside of cardiac muscle and calculates the measurement value for each local region of the cardiac muscle. The present embodiment is different from the first embodiment in that the measurement target is a 3-dimensional signal and a 3-dimensional image. Therefore, the difference from the first embodiment will be mainly described in this embodiment, by omitting the duplicative description as the first embodiment such as the apparatus configuration and the processing procedure.

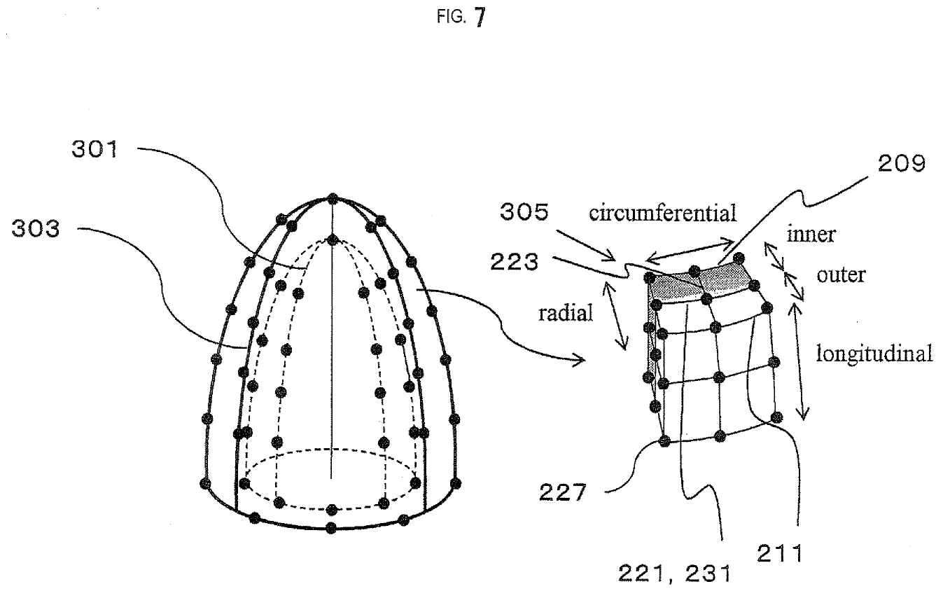

First, a 3-dimensional ultrasonic image is displayed on the basis of the ultrasonic signals of the biological tissue in plural cross sections of an object (S101), and the setting of a region of interest is carried out (S102). Here, the region of interest may be manually set on a 3-dimensional ultrasonic image using an input device, or may automatically set using a conventional automatic region segmentation method. In this manner, the setting of a 3-dimensional region of interest is executed. FIG. 7 is a view showing an example of the measurement method in a 3-dimensional ultrasonic image, and the left part of FIG. 7 shows the extracted view of an endocardium contour 301 and an epicardium contour 303 of the entire left ventricle. The right part of FIG. 7 is the extracted view of a certain 3-dimensional region of interest 305 which is set on a 3-dimensional ultrasonic image. As shown in the diagram, the cardiac muscle has, for example a 10 mm thick campanulate shape.

In the setting of a meshed pattern (S103), the segmentation is performed in the three directions of the radial direction, short-axis direction and the long-axis direction. That is, the objective of this segmentation method is to perform measurement in the respective radial, circumferential and longitudinal directions. In other words, in a certain cross section which is orthogonal to the longitudinal direction, the region of interest is segmented into a meshed pattern by a plurality of first segmenting lines 221 along the epicardiam 211 and the endocardium 209 of the cardiac muscle and a plurality of second segmenting lines 223 that are orthogonal to the plurality of first segmenting lines 221. Further, by elongating the first segmenting lines 221 and the second segmenting lines 223 along the epicardiam and the endocardium in the longitudinal direction and cutting the cross section that are orthogonal to the longitudinal direction at predetermined intervals in the direction thereof, the region of interest is segmented into a meshed pattern. The intersecting points of these segmenting lines are set as measurement points 227.

Also, by setting of a region-of-interest segmenting line 231 (S104), the 3-dimensional region of interest is segmented into local regions. For example, as shown in the right part of FIG. 7, the region-of-interest segmenting line 231 is set for dividing the region of interest into the endocardium side and the epicardium side. Here, after the region-of-interest segmenting line 231 is set, the 3-dimensional region of interest is segmented into a 3-dimensional region on the endocardiac side and a 3-dimensional region on the epicardiac side by the plane of which the region-of-interest segmenting line 231 is elongated along the endocardiam and the epicardium in the longitudinal direction.

Next, the apparatus executes the measurement of movement with respect to the respective measurement points 227 in the 3-dimensional signal and the 3-dimensional ultrasonic image (S105), and calculates the physical quantity (S106). The physical quantity is the distance in the respective radial, circumferential and longitudinal directions, the strain based on the distance variation, the area of a certain cross section in a 3-dimensional region of interest, the volume of a 3-dimensional region of interest, and so on. The examiner or the apparatus selects a result display region on the basis of the self-evaluation of movement tracking (S107). In the display of measurement result (S108), the measurement values are converted into luminance values, and the surface of the region of interest is colored. Then the examiner adjusts the position of the region-of-interest segmenting line while viewing the graph which indicates the result of movement tracking (S109).

As described above, in accordance with the present embodiment, since measurement values are appropriately set in conformity to the 3-dimensional property or extraction and contraction of cardiac muscle, it is possible to measure expansion and contraction of cardiac muscle which facilitates accurate reflection of expansion and contraction of cardiac muscle to the respective measurement values for making a proper diagnosis, as in the first embodiment. Also, the setting of a region of interest can be coincided with the property of biological tissue in a 3-dimensional space, which enables the tracking of movement inside of the biological tissue. Addition of the depth dimension in measurement also enables simultaneous measurement in three directions, which improves measurement accuracy compared to the 2-dimensional analysis.

Moreover, by separating a region of interest by a region-of-interest segmenting line and setting it as a local region or pseudocoloring the measurement values, the difference of local properties in the biological tissue can be easily discriminated.

The ultrasonic diagnostic apparatus of the present embodiment comprises in its basic configuration:

an ultrasonic probe configured to transmit/receive ultrasonic waves to/from an object;

an ultrasonic signal generating unit configured to generate an ultrasonic signal on the basis of the reflected echo signal, which is received by the ultrasonic probe, in the cross section of the tissue including the cardiac muscle of the heart of the object;

an ultrasonic image constructing unit configured to construct an ultrasonic image on the basis of ultrasonic signals;

a display unit configured to display the ultrasonic image;

a measurement position setting unit configured to set a region of interest in the cardiac muscle on the ultrasonic image displayed on the display unit;

a tracking calculation unit configured to track the movement of cardiac muscle in a plurality of measurement points in a region of interest; and

a physical quantity calculating unit configured to calculate a specific physical quantity on the basis of the tracking result,

and displays the calculated physical quantity on the display unit. As for a specific physical quantity, at least one of the myocardial velocity in plural measurement points of a region of interest, the strain of the cardiac muscle in a plurality of measurement points in a region of interest, the area of cardiac muscle which is framed by a region of interest and, in the case that a 3-dimensional ultrasonic image is generated, the volume of cardiac muscle which is surrounded by a 3-dimensional region of interest can be cited.

In order to solve the previously described problem, the tracking calculation unit is characterized in tracking the plurality of measurement points by a plurality of first segmenting line along the epicardium and the endocardium of the cardiac muscle and the second segmenting lines that are orthogonal to the first segmenting lines. For example, it may be configured so that plural measurement points are set on the basis of plural first segmenting lines and plural second segmenting lines, so that the set plural measurement points are tracked for measurement. More specifically, the region of interest is segmented into a meshed pattern by plural first segmenting lines and plural second segmenting lines, for setting the intersecting points of the first segmenting lines and the second segmenting lines as plural measurement points.

In order to improve accuracy in diagnosis of a specific physical quantity of cardiac muscle, proper setting of measurement points in conformity to the property or the expanding/contracting direction of the cardiac muscle is necessary. In this regard, the present invention sets plural measurement points along the main expanding and contracting direction of the cardiac muscle (for example, if an ultrasonic image shows the cardiac short axis view of an object including the heart chamber and the cardiac muscle surrounding the heart chamber forming a doughnut shape, the circumferential direction and the irradiating direction (radial direction) of the cardiac muscle). In this manner, since measurement points are set along the expanding and contracting direction of the cardiac muscle, in the case, that the distance variation between the adjacent measurement points on a first segmenting line or the adjacent measurement points on a second segmenting line and the like is measured, the property of the respective regions of the cardiac muscle is adequately reflected to the respective measurement points, which makes the measurement values useful for making an accurate diagnosis. Also, all of the distances from the endocardium or the epicardium to the plurality of measurement points that are respectively set on a plurality of first segmenting lines become constant. Therefore, in the case, for example that the distance variation from the respective measurement points on a certain first segmenting line to the endocardium or the epicardium is measured, the property of the respective regions in the cardiac muscle can be appropriately compared by comparing the measurement values, which is useful for making an accurate diagnosis.

Also, in the case an ultrasonic image to be displayed on the display unit is a cardiac short axis view, the measurement position setting unit can set a region of interest in a doughnut shape in conformity to the property of the doughnut-shaped cardiac muscle in the short axis view of the heart. On the other hand, the measurement position setting unit can set a plurality of regions of interest by separating them to a plurality of partial regions in the doughnut-shaped cardiac muscle of the cardiac short axis view. It is preferable to set plural regions of interest in this manner for comparing and studying specific physical quantities in the respective regions of interest. In this manner, this method of dividing a region of interest into plural separated regions is preferable for performing contrast observation of the specific physical quantities in the respective regions of interest. The measurement position setting unit is also capable of segmenting the doughnut-shaped cardiac muscle in the short axis view of the heart into the cardiac muscle on the side which is close to the transmitting/receiving surface of an ultrasonic probe and the cardiac muscle on the far side with the heart chamber interposed therebetween as separate regions of interests.

In the cardiac short axis view, the image quality of an ultrasonic image is not sufficient due to influence of noise, etc. in cardiac regions of a doughnut-shaped cardiac muscle other than the region which is close to the transmitting/receiving surface of the ultrasonic probe and the region which is away from the surface with a heart chamber interposed therebetween, which can lower the accuracy of the movement tracking. Therefore, even when the physical quantity is calculated in these cardiac muscle regions, the calculated values may lack credibility. If the region of interest is set as a doughnut-shaped pattern despite of the above-described tendency, the measurement will be executed even in the regions which lack credibility, which is not preferable in view of calculation efficiency. In view of this, by separating the doughnut-shaped cardiac muscle in a cardiac short axis view and setting a region which is close to the transmitting/receiving surface of an ultrasonic probe and a region which is away from the surface with the heart chamber interposed therebetween, it is possible to improve the efficiency in calculation process and to enable contrast observation of the region which is close to the transmitting/receiving surface of the ultrasonic probe and the region which is away from the surface with the heart chamber interposed therebetween.

Also, the measurement position setting unit is capable of setting one of the plurality of first segmenting lines or the plurality of second segmenting lines that are set in a region of interest as a region-of-interest segmenting line for dividing the region of interest into two regions, or setting a region-of-interest segmenting line, on an ultrasonic image, which is separate from the plurality of first segmenting lines or the plurality of second segmenting lines. In this manner, it is possible to easily divide a region of interest which is once set, which facilitates the convenience in evaluating the measurement values of endocardiac side and the epicardiac side of the cardiac muscle separately or evaluating the normal region and the abnormal region separately.

### DESCRIPTION OF REFERENCE NUMERALS

1: object
3: ultrasonic probe
5: ultrasonic signal generating unit
7: ultrasonic image generating unit
11: input unit
13: display unit
15: control unit
17: measurement position setting unit
19: tracking calculation unit
21: physical quantity calculating unit
100: ultrasonic diagnostic apparatus
203: ultrasonic image
205: heart chamber
207: cardiac muscle
209: endocardium
211: epicardium
213: first region of interest
215: second region of interest
221: first segmenting line
223: second segmenting line
227: measurement point
231: region-of-interest segmenting line
305: 3-dimensional region of interest

1. An ultrasonic diagnostic apparatus comprising:
an ultrasonic probe configured to transmit/receive ultrasonic waves to/from an object to be examined;
an ultrasonic signal generating unit configured to generate an ultrasonic signal on the basis of the reflected echo signal, which is received by the ultrasonic probe, in the cross section of the tissue including the cardiac muscle of the heart of the object;
an ultrasonic image generating unit configured to generate an ultrasonic image on the basis of the ultrasonic signal;
a display unit configured to display the ultrasonic image;
a measurement position setting unit configured to set a region of interest in the cardiac muscle on the ultrasonic image displayed on the display unit;
a tracking calculation unit configured to track the movement of cardiac muscle in a plurality of measurement points in the region of interest; and
a physical quantity calculating unit configured to calculate a specific physical quantity on the basis of the tracking result,
wherein:
the calculated physical quantity is displayed on the display unit; and
the tracking calculation unit tracks the plurality of measurement points by a plurality of first segmenting lines along the epicardium and the endocardium of the cardiac muscle and a plurality of second segmenting lines that are orthogonal to the plurality of first segmenting lines.

2. The ultrasonic diagnostic apparatus according to claim 1, wherein:
the measurement position setting unit segments the region of interest by the plurality of first segmenting lines along the epicardium and the endocardium of the cardiac muscle and the plurality of second segmenting lines that are orthogonal to the plurality of first segmenting lines; and
sets the intersecting points of the first segmenting lines and the second segmenting lines as the plurality of measurement points.

3. The ultrasonic diagnostic apparatus according to claim 1, wherein the measurement position setting unit, in the case that the ultrasonic image to be displayed on the display unit is a cardiac short axis view of the object including the heart chamber and the cardiac muscle surrounding the heart chamber, is configured capable of setting the region of interest in conformity to the property of the cardiac muscle in the cardiac short axis view.

4. The ultrasonic diagnostic apparatus according to claim 1, wherein the measurement position setting unit, in the case that the ultrasonic image to be displayed on the display unit is a cardiac short axis view of the object including the heart chamber and the cardiac muscle surrounding the heart chamber, is configured capable of setting a region of interest by segmenting the cardiac muscle in the cardiac short axis view into plural partial regions.

5. The ultrasonic diagnostic apparatus according to claim 1, wherein the measurement position setting unit, in the case that the ultrasonic image to be displayed on the display unit is a cardiac short axis view of the object including the heart chamber and the cardiac muscle surrounding the heart chamber, is configured capable of setting the region of interest by separating the cardiac muscle in the cardiac short axis view into the cardiac muscle which is on the side close to the transmitting/receiving surface of the ultrasonic probe and the cardiac muscle which is on the side away from the transmitting/receiving surface, with the heart chamber interposed therebetween.

6. The ultrasonic diagnostic apparatus according to claim 1, wherein the measurement position setting unit is configured capable of setting one of the plurality of first segmenting lines or one of the plurality of second segmenting lines that are set in the region of interest as a region-of-interest segmenting line for dividing the region of interest into two regions, or setting the region-of-interest segmenting line on an ultrasonic image separately from the plurality of first segmenting lines or the plurality of second segmenting lines.

7. The ultrasonic diagnostic apparatus according to claim 1, wherein:
the ultrasonic probe has configuration in which a plurality of transducers that transmit/receive ultrasonic waves to/from the object are two-dimensionally arrayed or the plurality of transducers are one-dimensionally arrayed for mechanically executing spatial scanning so as to measure the reflected echo signals in plural cross sections of the tissue including the cardiac muscle of the heart of the object;
the display unit displays the 3-dimensional ultrasonic image generated on the basis of the ultrasonic signals in the plural cross sections; and
the measurement position setting unit sets a 3-dimensional region of interest on the 3-dimensional ultrasonic image displayed on the display unit.

8. The ultrasonic diagnostic apparatus according to claim 1, wherein the specific physical quantity is at least one of the myocardial velocity in a plurality of measurement points in the region of interest, the strain of the cardiac muscle in the plurality of measurement points in the region of interest, the area of the cardiac muscle which is framed by the region of interest, and the volume of the cardiac muscle which is framed by the 3-dimensional region of interest.

9. A measurement point tracking method of the ultrasonic diagnostic apparatus which:
transmits/receives ultrasonic waves to/from an object to be examined by an ultrasonic probe;
generates an ultrasonic signal by an ultrasonic signal generating unit on the basis of the reflected echo signal, which is received by the ultrasonic probe, in the cross section of the tissue including the cardiac muscle of the heart of the object;
generates an ultrasonic image by an ultrasonic image generating unit on the basis of the ultrasonic signal;
displays the ultrasonic image by a display unit;
sets a region of interest by a measurement position setting unit in the cardiac muscle on the ultrasonic image which is displayed on the display unit;
tracks the movement of cardiac muscle in a plurality of measurement points in the region of interest by a tracking calculation unit;
calculates a specific physical quantity by a physical quantity calculating unit on the basis of the tracking result; and
displays the calculated physical quantity on the display unit,
including a step of tracking the plurality of measurement points by the tracking calculation unit using a plurality of first segmenting lines along the epicardium and the endocardium of the cardiac muscle and a plurality of second segmenting lines that are orthogonal to the plurality of first segmenting lines.

10. The measurement point tracking method of the ultrasonic diagnostic apparatus according to claim 9, wherein:
the region of interest is segmented by the plurality of first segmenting lines along the epicardium and the endocardium of the cardiac muscle and the plurality of second segmenting lines that are orthogonal to the plurality of first segmenting lines; and
the intersecting points of the first segmenting lines and the second segmenting lines are set as the plurality of measurement points.

11. The measurement point tracking method of the ultrasonic diagnostic apparatus according to claim 9, wherein the measurement position setting unit, in the case that the ultrasonic image to be displayed on the display unit is a cardiac short axis view of the object including the heart chamber and the cardiac muscle surrounding the heart chamber, is configured capable of setting the region of interest in conformity to the property of the cardiac muscle in the cardiac short axis view.

12. The measurement point tracking method of the ultrasonic diagnostic apparatus according to claim 9, wherein the region of interest, in the case that the ultrasonic image to be displayed on the display unit is a cardiac short axis view of the object including the heart chamber and the cardiac muscle surrounding the heart chamber, can be set by the measurement position setting unit by being segmented into plural partial regions in the cardiac muscle in the cardiac short axis view.

13. The measurement point tracking method of the ultrasonic diagnostic apparatus according to claim 9, wherein the region of interest in the cardiac muscle in the cardiac short axis view, in the case that the ultrasonic image to be displayed on the display unit is a cardiac short axis view of the object including the heart chamber and the cardiac muscle surrounding the heart chamber, can be set by the measurement position setting unit by being segmented into the cardiac muscle which is on the side close to the transmitting/receiving surface of the ultrasonic probe and the cardiac muscle which is on the side away from the transmitting/receiving surface, with the heart chamber interposed therebetween.

14. The measurement point tracking method of the ultrasonic diagnostic apparatus according to claim 9, wherein one of the plurality of first segmenting lines or the plurality of second segmenting lines that are set in the region of interest for dividing the region of interest into two regions can be set as a region-of-interest segmenting line, or a region-of-interest segmenting line can be set on an ultrasonic image separately from the plurality of first segmenting lines or the plurality of second segmenting lines.

15. The measurement point tracking method of the ultrasonic diagnostic apparatus according to claim 9, wherein:
the ultrasonic probe has configuration in which a plurality of transducers that transmit/receive ultrasonic waves to/from the object are two-dimensionally arrayed or the plurality of transducers are one-dimensionally arrayed for mechanically executing spatial scanning so as to measure the reflected echo signals in plural cross sections of the tissue including the cardiac muscle of the heart of the object;
the 3-dimensional ultrasonic image generated on the basis of the ultrasonic signal in the plural cross sections is displayed on the display unit; and
a 3-dimensional region of interest is set by the measurement position setting unit on the 3-dimensional ultrasonic image displayed on the display unit.
